(19) United States
(12) Patent Application Publication
(10) Pub. No.: US 2023/0313273 A1
(43) Pub. Date: Oct. 5, 2023

(54) SELECTING APTAMERS USING SEQUENCING

(71) Applicant: Illumina, Inc., San Diego, CA (US)

(72) Inventor: Cassandra Chamoun, San Mateo, CA (US)

(73) Assignee: Illumina, Inc., San Diego, CA (US)

(21) Appl. No.: 18/000,666

(22) PCT Filed: Dec. 16, 2021
(86) PCT No.: PCT/US2021/063835

§ 371 Date: Dec. 2, 2022

Related U.S. Application Data
(60) Provisional application No. 63/128,669, filed on Dec. 21, 2020.

(51) Int. Cl. C12Q 1/6811; C12Q 1/6869; C12Q 1/686

(52) U.S. Cl. CPC C12Q 1/6811; C12Q 2600/16; C12Q 1/686; C12Q 1/6869

(57) ABSTRACT

Methods and systems for selecting an aptamer from a plurality of aptamer candidates are provided. A target is coupled within each well of a plurality of wells that is disposed within a substrate. Each of the wells is contacted with a fluid comprising a plurality of aptamer candidates. Within at least one of the wells, any aptamer candidate that is selective for the target is coupled to the target. Any aptamer candidates that are not coupled to the target are removed. Within the wells, any aptamer candidates are sequenced that remain after the removing to identify any aptamers that are selective for the target.

### CROSS-REFERENCE TO RELATED APPLICATIONS

This application claims the benefit of U.S. Provisional Patent Application No. 63/128,669, filed Dec. 21, 2020 and entitled “Selecting Aptamers Using Sequencing,” the entire contents of which are incorporated by reference herein.

### BACKGROUND

The detection of specific nucleic acid sequences, which may be present in a biological sample, has been used as a method for identifying and classifying microorganisms, diagnosing infectious diseases, detecting and characterizing genetic abnormalities, identifying genetic changes associated with cancer, studying genetic susceptibility to diseases, and measuring response to various types of treatment, as some examples. Nucleic acid sequences that bind to a specific target molecule may be referred to as an “aptamer,” and may be synthetic or may come from a biological sample. A common technique for detecting specific nucleic acid sequences, whether synthetic or from a biological sample, is nucleic acid sequencing.

Nucleic acid sequencing methodology has evolved from the chemical degradation methods used by Maxam and Gilbert and the strand elongation methods used by Sanger. Several sequencing methodologies are now in use which allow for the parallel processing of millions, or even billions, of nucleic acids on a single flow cell. Some platforms include bead-based and microarray formats in which silica beads are functionalized with probes depending on the application of such formats in applications including sequencing, genotyping, or gene expression profiling. Some sequencing systems, whether for “sequencing-by-synthesis” or for genotyping, utilize substrates including a plurality of different reservoirs that carry different reagents for use in sequencing operations.

### SUMMARY

Examples provided herein are related to selecting aptamers using sequencing.

Apparatus and methods for performing such selection are disclosed.

Some examples herein provide a method of selecting an aptamer from a plurality of aptamer candidates. The method may include coupling a target within each well of a plurality of wells that is disposed within a substrate. The method may include contacting each of the wells with a fluid including a plurality of aptamer candidates. The method may include, within at least one of the wells, coupling to the target any aptamer candidate that is selective for the target. The method may include removing any aptamer candidates that are not coupled to the target. The method may include sequencing, within the wells, any aptamer candidates that remain after the removing to identify any aptamers that are selective for the target.

In some examples, coupling the target within each well of the plurality of wells includes coupling a first moiety within each of the wells; coupling a plurality of second moieties to respective ones of the target; and coupling the second moiety to the first moiety within each of the wells. In some examples, the first moiety includes streptavidin and the second moiety includes biotin. In some examples, the first moiety is coupled within each of the wells by a capture primer. In some examples, detaching the first moiety from the capture primer before performing the sequencing.

In some examples, the substrate includes detection circuitry used to sequence the aptamer candidates that remain after the removing.

In some examples, the plurality of wells include flow cells through which the fluid is flowed in parallel.

In some examples, any aptamer candidate that is selective for the target has a tertiary structure that changes when becoming coupled to the target.

In some examples, the method further includes generating amplicons of any aptamer candidate that is selective for the target. In some examples, generating amplicons of any aptamer candidate that is selective for the target includes: decoupling that aptamer candidate from the target; and using polymerase chain reaction (PCR) to generate the amplicons using that aptamer candidate. In some examples, the PCR is performed in an amplification chamber that is distinct from the wells. In some examples, the method further includes contacting each of the wells with a fluid including the amplicons; within each of the wells, coupling to the target any amplicon that is selective for the target; and removing any amplicons are not coupled to the target. In some examples, any aptamer candidates that are sequenced include any amplicons that remain after removing any amplicons that are not coupled to the target. In some examples, the method further includes generating additional amplicons of any amplicon that is selective for the target.

In some examples, sequencing any aptamer candidates includes: coupling, to capture primers disposed within the wells, any aptamer candidates that remain after the removing; performing amplification within the wells to generate amplicons coupled to the capture primers; and sequencing the amplicons coupled to the capture primers. In some examples, the target is coupled within the wells via respective ones of the capture primers. In some examples, the capture primers couple a first moiety within the wells, and a second moiety is coupled to the first moiety and to the target within the wells. In some examples, the capture primers are coupled to the well separately from the target. In some examples, each of the aptamer candidates includes first and second adapters that are complementary to respective capture primers. In some examples, each of the aptamer candidates further includes a first spacer disposed between the first adapter and a region that is a candidate to be selective for the target, and a second spacer disposed between the second adapter and the region that is a candidate to be selective for the target.

In some examples, each of the aptamer candidates includes an oligonucleotide.

Some examples herein provide a system for selecting an aptamer from a plurality of aptamer candidates. The system may include a substrate including a plurality of wells. The system may include a target coupled within each of the wells. The system may include a fluid including a plurality of aptamer candidates and contacting each of the wells, wherein any aptamer candidate that is selective for the target becomes coupled to the target. The system may include detection circuitry to sequence any aptamer candidates within the wells to identify any aptamers that are selective for the target.

In some examples, a first moiety is coupled within each of the wells. In some examples, a plurality of second moieties are coupled to respective ones of the target. In some examples, the second moiety is coupled to the first moiety within each of the wells to couple the target within each of the wells. In some examples, the first moiety includes streptavidin and the second moiety includes biotin. In some examples, the first moiety is coupled within each of the wells by a capture primer. In some examples, the first moiety is detachable from the capture primer.

In some examples, the plurality of wells are disposed on the detection circuitry.

In some examples, the plurality of wells include flow cells through which the fluid is flowed in parallel.

In some examples, any aptamer candidate that is selective for the target has a tertiary structure that changes when becoming coupled to the target.

In some examples, amplicons are generated of any aptamer candidate that is selective for the target. In some examples, the amplicons of any aptamer candidate that is selective for the target are generated using steps including: decoupling that aptamer candidate from the target; and using polymerase chain reaction (PCR) to generate the amplicons using that aptamer candidate. In some examples, the system includes an amplification chamber that is distinct from the wells to perform the PCR.

In some examples, the system further includes a fluid including the amplicons and contacting each of the wells, wherein any amplicon that is selective for the target becomes coupled to the target. In some examples, any aptamer candidates that are sequenced include any amplicons that remain after removing any amplicons that are not coupled to the target. In some examples, the system further includes additional amplicons of any amplicon that is selective for the target.

In some examples, the system further includes capture primers disposed within the wells and coupled to any aptamer candidates that remain after the removing; and amplicons of the aptamer candidates that are coupled to the capture primers. The detection circuitry may be to sequence the amplicons coupled to the capture primers. In some examples, the target is coupled within the wells via respective ones of the capture primers. In some examples, the capture primers couple a first moiety within the wells, and a second moiety is coupled to the first moiety and to the target within the wells. In some examples, the capture primers are coupled to the well separately from the target. In some examples, each of the aptamer candidates includes first and second adapters that are complementary to respective capture primers. In some examples, each of the aptamer candidates further includes a first spacer disposed between the first adapter and a region that is a candidate to be selective for the target, and a second spacer disposed between the second adapter and the region that is a candidate to be selective for the target.

In some examples, each of the aptamer candidates includes an oligonucleotide.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1I schematically illustrate example an example apparatus and operations used in a process flow for selecting aptamers using sequencing.

FIGS. 2A-2F schematically illustrate an example apparatus and operations used in an alternative process flow for selecting aptamers using sequencing.

FIG. 3 schematically illustrates example aptamer candidates for use in an apparatus or process flow such as described with reference to FIGS. 1A-1I or 2A-2F.

FIG. 4 schematically illustrates example operations in a process flow for selecting aptamers using sequencing.

### DETAILED DESCRIPTION

Examples provided herein are related to selecting aptamers using sequencing. Apparatus and methods for performing such selection are disclosed.

Some aptamers are oligonucleotide strands that bind with high selectivity to target molecules, such as enzymes, antibodies, single cells, or any other molecular target of interest. During such selective binding, the aptamers may obtain a tertiary structure. SELEX, or Systematic Evolution of Ligands by EXponential Enrichment, is a process by which aptamers are selected. Previously known SELEX processes are typically performed on a conventional solid phase substrate, such as a column containing a medium to which a target is coupled. A fluid including aptamer candidates is flowed through the substrate. Any aptamers that are selective for the target become coupled to the target while the other aptamers are not coupled and thus flow out of the substrate. The aptamers that are selective for the target then are decoupled from the target and amplified using polymerase chain reaction (PCR), e.g., in 96-well plates. The PCR products again may be flowed through the substrate and amplified, to provide further enrichment. The amplified aptamers then are sequenced to identify the aptamers which became coupled to the target and thus are the most “successful.” These processes can be time and labor intensive.

In comparison, as provided herein, the present apparatus and methods may streamline and simplify the SELEX process through use of sequencing. More specifically, in some examples, operations for both selecting and sequencing aptamers may be performed within the same wells, e.g., within the wells of a substrate within a sequencing system. The substrate upon which the selection process is performed may include detection circuitry for use in sequencing the aptamers. Illustratively, a target may be coupled within each of the wells, and a plurality of aptamers flowed into each of the wells. Any aptamers that are selective for the target may become coupled to the target, and any other aptamers may be removed. The aptamers that are selective for the target may be amplified, and then may be sequenced within the wells, e.g., using capture primers that are also coupled within each of the wells. As such, the present apparatus and methods may provide a significantly streamlined and simplified process with significantly less material usage and waste as compared to previously known SELEX processes.

First, some terms used herein will be briefly explained. Then, some example methods for selecting aptamers using sequencing, and associated apparatus, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarily between the sequences of nucleotides within those polynucleotides. The strength of hybridization between polynucleotides may be characterized by a temperature of melting (Tm) at which 50% of the duplexes disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Example polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” refers to a polynucleotide to which nucleotides may be added via a free 3′ OH group. The primer length may be any suitable number of bases long and may include any suitable combination of natural and non-natural nucleotides. A target polynucleotide (such as, but not limited to, an aptamer) may include an “adapter” that hybridizes to (has a sequence that is complementary to) a primer, and may be amplified so as to generate a complementary copy polynucleotide by adding nucleotides to the free 3′ OH group of the primer. A primer may be coupled to a substrate. Primers that are “complementary” to one another may hybridize to one another along substantially their entire lengths, whereas primers that are “orthogonal” with one another substantially do not hybridize with one another, nor do their amplicons. A “capture primer” is intended to mean a primer that is coupled to the substrate and may hybridize to a first adapter of a target polynucleotide, while an “orthogonal capture primer” is intended to mean a primer that is coupled to the substrate and may hybridize to a second adapter of that target polynucleotide. The first adapter may have a sequence that is complementary to that of the capture primer, and the second adapter may have a sequence that is complementary to that of the orthogonal capture primer. A capture primer and an orthogonal capture primer may have different and independent sequences than one another.

In some examples, capture primers are P5 or P7 primers that are commercially available from Illumina, Inc. P5 and P7 primers are nonlimiting examples of primers that are orthogonal to one another. The P5 and P7 primer sequences may have the following sequences, in some examples:

Paired read set:
P5: 5′-AATGATACGGCGACCACCGAGAUCTACAC-3′
P7: 5′-CAAGCAGAAGACGGCATACGAG*AT-3′
Single read set:
P5: 5′-AATGATACGGCGACCACCGA-3′
P7: 5′-CAAGCAGAAGACGGCATACGA3′
where G* is G or 8-oxoguanine.

In some examples, the attached oligonucleotides (such as primers or P5 or P7 primers) include a linker or spacer at the 5′ end. Such linker or spacer may be included in order to permit chemical or enzymatic cleavage, or to confer some other desirable property, for example to enable covalent attachment to a polymer or a solid support, or to act as spacers to position the site of cleavage an optimal distance from the solid support. In certain cases, 10 spacer nucleotides may be positioned between the point of attachment of the P5 or P7 primers to a polymer or a solid support. In some examples, polyT spacers are used, although other nucleotides and combinations thereof can also be used. In one example, the spacer is a 6 T to 10 T spacer. In some examples, the linkers include cleavable nucleotides including a chemically cleavable functional group such as a vicinal diol or allyl T.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of wells (e.g., microwells or nanowells) on glass, silicon, plastic or other suitable material(s) with patterned, covalently-linked gel such as poly(N-(5-azidoacetamidylpentyl) acrylamide-co-acrylamide) (PAZAM). The process creates gel pads used for sequencing that may be stable over sequencing runs with a large number of cycles. The covalent linking of the polymer to the wells may be helpful for maintaining the gel in the structured features throughout the lifetime of the structured substrate during a variety of uses. However in many examples, the gel need not be covalently linked to the wells. For example, in some conditions silane free acrylamide (SFA) which is not covalently attached to any part of the structured substrate, may be used as the gel material.

In particular examples, a structured substrate may be made by patterning a suitable material with wells (e.g. microwells or nanowells), coating the patterned material with a gel material (e.g., PAZAM, SFA or chemically modified variants thereof, such as the azidolyzed version of SFA (azido-SFA)) and polishing the surface of the gel coated material, for example via chemical or mechanical polishing, thereby retaining gel in the wells but removing or inactivating substantially all of the gel from the interstitial regions on the surface of the structured substrate between the wells. Primers may be attached to gel material. A solution including a plurality of target polynucleotides (e.g., a fragmented human genome or portion thereof) may then be contacted with the polished substrate such that individual target polynucleotides will seed individual wells via interactions with primers attached to the gel material; however, the target polynucleotides will not occupy the interstitial regions due to absence or inactivity of the gel material. Amplification of the target polynucleotides may be confined to the wells because absence or inactivity of gel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An example patterned substrate having these characteristics is the etched substrate used in connection with BEAD ARRAY technology (Illumina, Inc., San Diego, Calif.).

In some examples, a substrate forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and systems set forth herein include, but are not limited to, those commercially available from Illumina, Inc. (San Diego, CA).

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “sequencing system” refers to a system that is configured to determine the sequence of polynucleotides. A variety of sequencing systems are commercially available. Illustratively, a sequencing system may be or include the iSEQ™ 100 Sequencing System, commercially available from Illumina, Inc. (San Diego, CA). The iSEQ™ 100 Sequencing System is a benchtop system that performs sequencing-by-synthesis using a prefilled cartridge that includes reservoirs storing different sequencing reagents. Other nonlimiting examples of sequencing systems include the cBot 2, NovaSeq 6000, and MiniSeq systems commercially available from Illumina, Inc., as well as sequencing systems from other sources.

As used herein, an “aptamer” is intended to mean an oligonucleotide that has a tertiary structure causing that oligonucleotide selective for a target, while an “aptamer candidate” is intended to mean an oligonucleotide that potentially may be selective for a target. To be “selective” for a target is intended to mean to couple to that target and not to couple to a different target. As such, among a given plurality of aptamer candidates, one or more aptamer candidates potentially may be selective for a given target and thus may be aptamers for that target. It will be appreciated, however, that any given plurality of aptamer candidates need not necessarily include an aptamer for a given target. An aptamer candidate may be identified as an aptamer that is selective for the target using operations including coupling the aptamer candidate to the target (indicating selectivity) and sequencing the aptamer candidate. An amplicon of an aptamer candidate may itself be an aptamer candidate, whether that amplicon is a copy or a complementary copy of the aptamer candidate. An amplicon of an aptamer may itself be an aptamer, e.g., where that amplicon is a copy of the aptamer (as opposed to a complementary copy of the aptamer, which complementary copy may be an aptamer candidate but not necessarily an aptamer). Aptamers may include any suitable type of oligonucleotide, e.g., DNA, RNA, and/or nucleic acid analogues such as exemplified elsewhere herein. An aptamer may become coupled to a target through any suitable combination of interactions, e.g., through any suitable combination of electrostatic interactions, hydrophobic interactions, and formation of a tertiary structure.

As used herein, “target” is intended to mean a chemical element for which it is desired to select an aptamer. Targets may include chemical entities that are not nucleotides. An example target is a protein target. A protein includes a sequence of polypeptides that are folded into a structure. Another example target is a metabolite target. A metabolite target is a chemical element that is formed or used during metabolism. Additional example targets include, but are not limited to, carbohydrates, fatty acids, sugars (such as glucose), amino acids, nucleosides, neurotransmitters, phospholipids, and heavy metals. In the present disclosure, analytes may be selected for targets in the context of any suitable application(s), such as analyzing a disease state, analyzing metabolic health, analyzing a microbiome, analyzing drug interaction, analyzing drug response, analyzing toxicity, or analyzing infectious disease. Illustratively, metabolites can include chemical elements that are upregulated or downregulated in response to disease. Nonlimiting examples of targets include kinases, serine hydrolases, metalloproteases, disease-specific biomarkers such as antigens for specific diseases, and glucose.

As used herein, an oligonucleotide with “tertiary structure” is intended to mean an oligonucleotide that is folded into a three-dimensional tertiary structure having internal cross-linking holding the folds in place. In comparison, an oligonucleotide that has a primary structure (e.g., a particular sequence of nucleic acids linked together) and a secondary structure (e.g., local structure) but no internal cross-linking holding folds into place would not be considered to have a tertiary structure as the term is used herein. The tertiary structure of the aptamer may change when the aptamer becomes coupled to the target.

As used herein, elements being “different” is intended to mean that one of the elements has at least one variation relative to the other element that renders the elements distinguishable one another. For example, oligonucleotides that are different than one another may have nucleic acid sequences that vary relative to one another by at least one nucleic acid. As another example, proteins that are different than one another may have peptide sequences that vary relative to one another by at least one peptide. As another example, metabolites may vary relative to one another by at least one chemical group. As provided herein, different aptamers may be selected for different targets using the present apparatus and methods.

As used herein, the term “fluorophore” is intended to mean a molecule that emits light at a first wavelength responsive to excitation with light at a second wavelength that is different from the first wavelength. The light emitted by a fluorophore may be referred to as “fluorescence” and may be detected by suitable optical detection circuitry.

Fluorescence may be detected using any suitable optical detection circuitry, which may include an optical detector to generate an electrical signal based on the light received from the fluorophore, and electronic circuitry to determine, using the electrical signal, that light was received from the fluorophore. As one example, the optical detector may include an active-pixel sensor (APS) including an array of amplified photodetectors configured to generate an electrical signal based on light received by the photodetectors. APSs may be based on complementary metal oxide semiconductor (CMOS) technology known in the art. CMOS-based detectors may include field effect transistors (FETs), e.g., metal oxide semiconductor field effect transistors (MOSFETs). In particular examples, a CMOS imager having a single-photon avalanche diode (CMOS-SPAD) may be used, for example, to perform fluorescence lifetime imaging (FLIM). In other examples, the optical detector may include a photodiode, such as an avalanche photodiode, charge-coupled device (CCD), cryogenic photon detector, reverse-biased light emitting diode (LED), photoresistor, phototransistor, photovoltaic cell, photomultiplier tube (PMT), quantum dot photoconductor or photodiode, or the like. The optical detection circuitry further may include any suitable combination of hardware and software in operable communication with the optical detector so as to receive the electrical signal therefrom, and configured to detect the fluorescence based on such signal, e.g., based on the optical detector detecting light from the fluorophore. For example, the electronic circuitry may include a memory and a processor coupled to the memory. The memory may store instructions for causing the processor to receive the signal from the optical detector and to detect the fluorophore using such signal. For example, the instructions can cause the processor to determine, using the signal from the optical detector, that fluorescence is emitted within the field of view of the optical detector and to determine, using such determination, that a fluorophore is present. A substrate may include optical detection circuitry, e.g., may include an optical detector upon which one or more wells may be disposed that are used for selecting aptamers using sequencing.

Selecting Aptamers Using Sequencing

As noted above and as described in greater detail below, the present apparatus and methods may be used to select aptamers using sequencing. A target may be coupled within each of a plurality of wells within which both aptamer selection and aptamer sequencing is to be performed. A plurality of aptamer candidates (e.g., a SELEX aptamer library) may be introduced to the wells to contact the targets with the aptamer candidates, and any aptamer candidate that is selective for the target becomes coupled to the target while the remaining aptamer candidates remain uncoupled to the target and may be washed away. The aptamer candidates which became coupled to the target may be amplified, and may be sequenced in the wells to identify the aptamer(s) that are selective for the target.

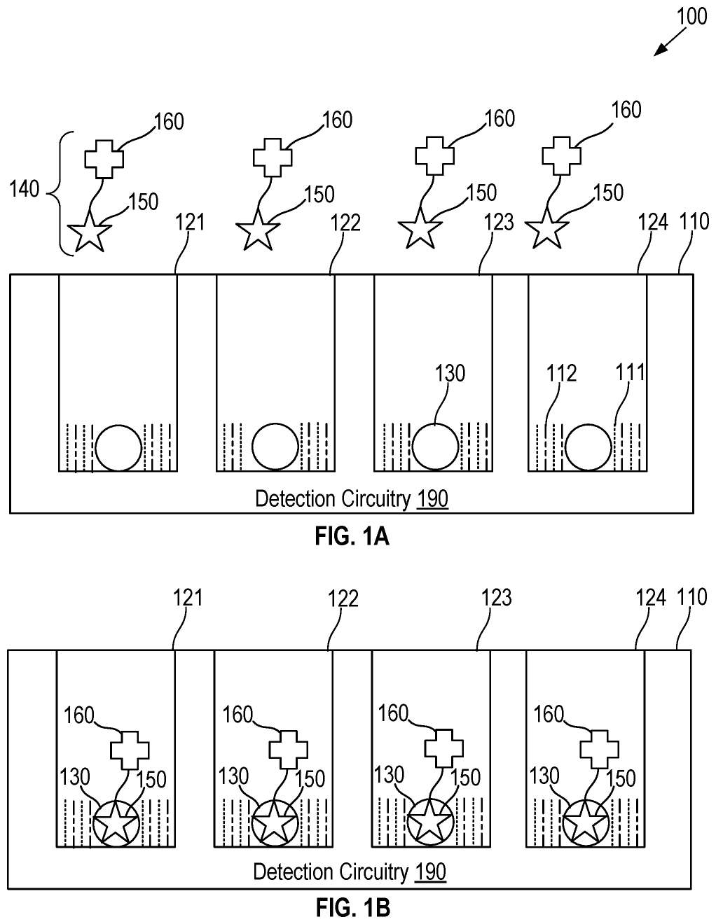

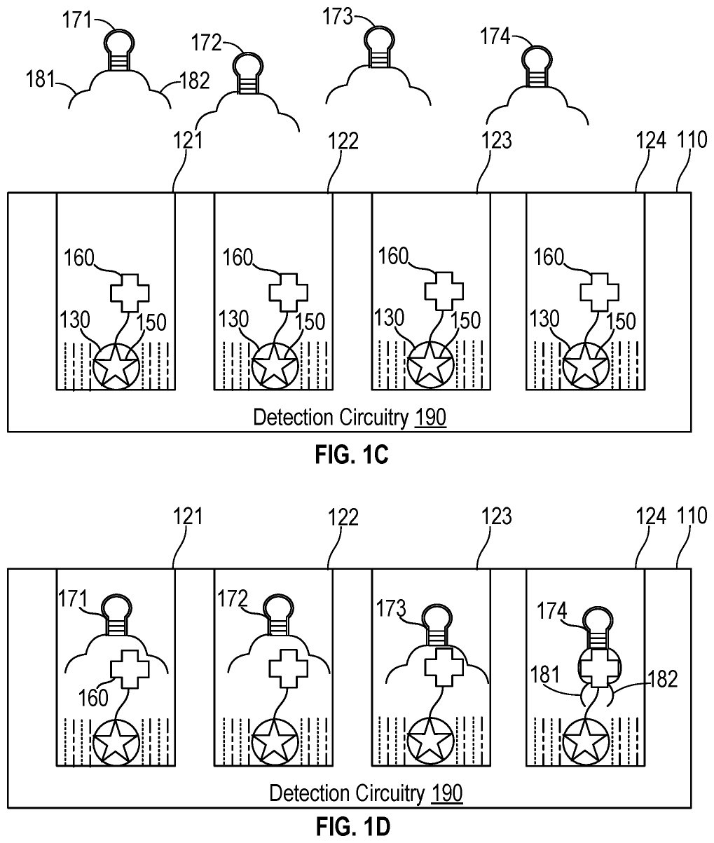

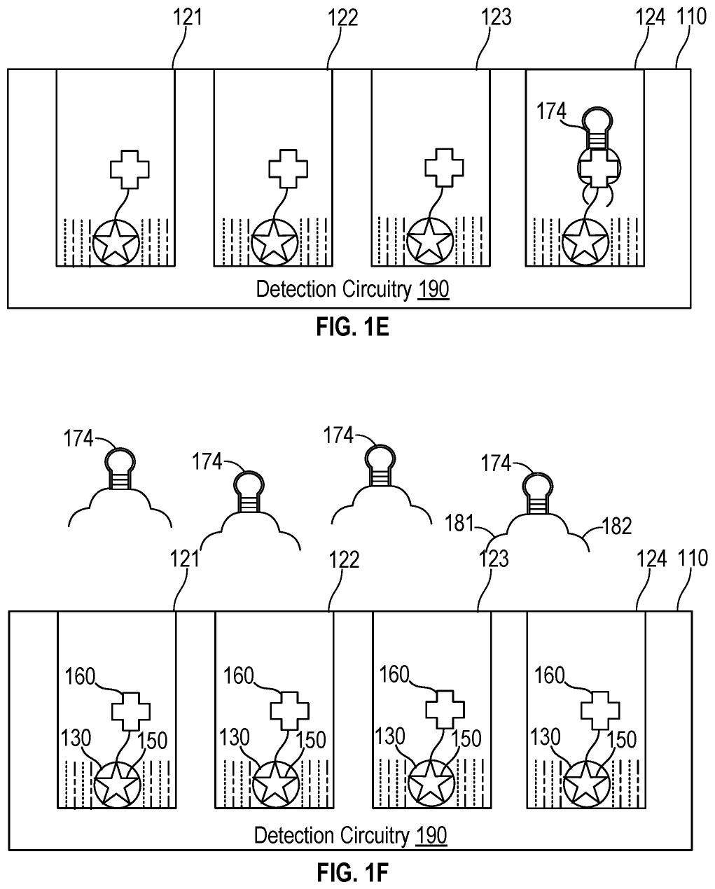

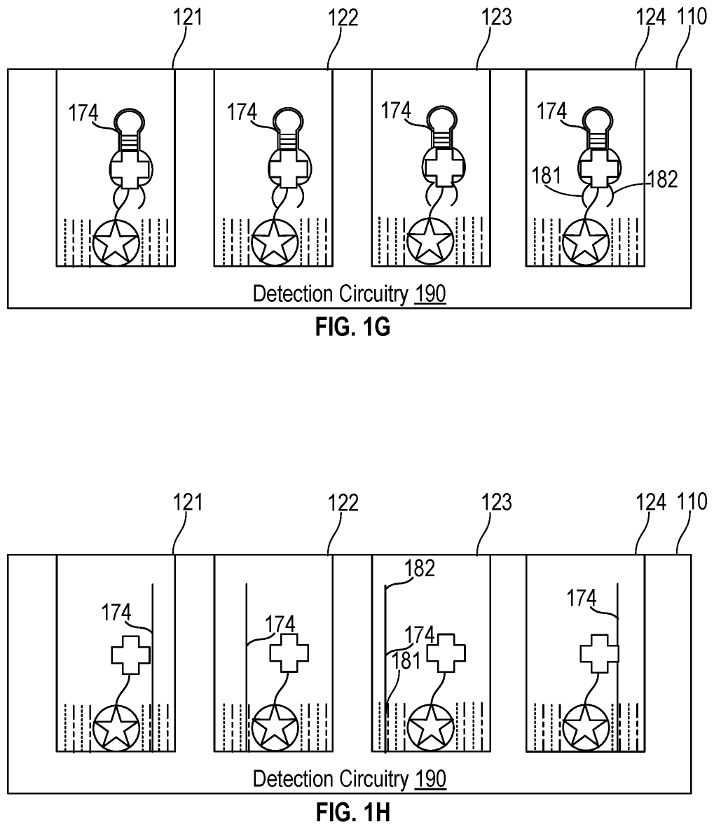

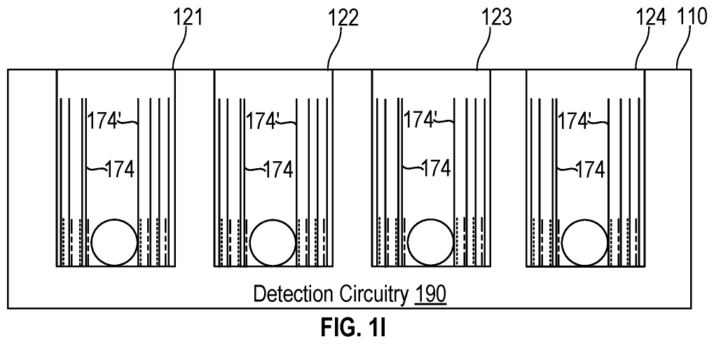

For example, FIGS. 1A-1I schematically illustrate example an example apparatus and operations used in a process flow for selecting aptamers using sequencing. Apparatus 100 illustrated in FIG. 1A may include a substrate 110 including comprising a plurality of reservoirs. In the nonlimiting example illustrated in FIG. 1A, reservoirs may include wells 121, 122, 123, 124 that are provided within a common, integrally formed substrate 110 as one another. However, it will be appreciated that one or more of wells 121, 122, 123, 124, and indeed all of wells 121, 122, 123, 124, may be physically separated from one another and need not be formed in a common substrate as one another. Any suitable number of wells of any suitable size and arrangement may be provided. For example, substrate 110 may include thousands, tens of thousands, hundreds of thousands, or even millions of wells. Each of wells 121, 122, 123, 124 may include a plurality of each of first and second capture primers 111, 112 that may be used to amplify and sequence candidate aptamers in a manner such as described further below with reference to FIGS. 1H-1I. Apparatus 100 further may include detection circuitry 190 to sequence any aptamer candidates within the wells to identify any aptamers that are selective for the target, e.g., in a manner such as described with reference to FIG. 1I. Wells 121, 122, 123, 124 may be disposed on or otherwise coupled to detection circuitry 190. In one nonlimiting example, detection circuitry 190 includes a CMOS optical detector, wells 121, 122, 123, 124 include flow cells through which fluid may be flowed in parallel, and the CMOS detects fluorescence during sequencing of oligonucleotides within the flow cells.

Illustratively, apparatus 100 may be for use in a sequencing system to select aptamers using sequencing. Illustratively, the sequencing system may be or include the iSEQ™ 100 Sequencing System, commercially available from Illumina, Inc. (San Diego, CA). The iSEQ™ 100 Sequencing System is a benchtop system that performs sequencing-by-synthesis using a prefilled cartridge that includes reservoirs storing different sequencing reagents in a manner similar to that of apparatus 100 illustrated in FIGS. 1A-1B. However, it will be appreciated that apparatus 100 suitably may be adapted for use with any other sequencing system, such as the cBot 2, NovaSeq 6000, or MiniSeq systems commercially available from Illumina, Inc., or a sequencing system from another source.

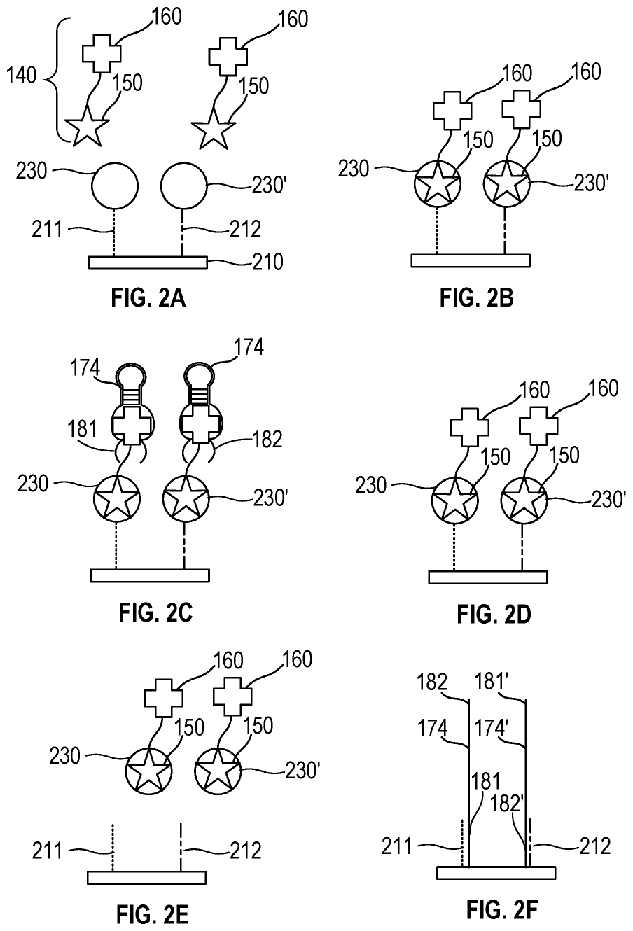

A target, for which it may be desired to find an aptamer, may be coupled within each of wells 121, 122, 123, 124. Nonlimiting examples of targets are provided elsewhere herein. The target may be directly coupled via any suitable linkage, e.g., via a capture primer in a manner such as described further below with reference to FIGS. 2A-2F. Alternatively, the target may be indirectly coupled via moieties that interact with one another. For example, in a manner such as illustrated in FIG. 1A, a first moiety 130 may be coupled within each of wells 121, 122, 123, 124, e.g., via any suitable linkage. The wells 121, 122, 123, 124 may be contacted with a fluid including a plurality of molecules 140, each of which molecules may include second moiety 150 and target 160 coupled to one another via any suitable linkage, which linkage optionally is cleavable. The respective linkages between first moiety 130 and wells 121, 122, 123, 124, and between second moiety 150 and target 160, may be formed through any suitable interactions such as NTA-His-Tag, Spytag-Spycatcher, hybridization of an oligonucleotide to a complementary oligonucleotide, copper(I)-catalyzed click reaction, or strain-promoted azide-alkyne cycloaddition. Any suitable number of such linkages may include a cleavable moiety, such as 8-oxo-G or a protein which may be cleaved using a proteinase, which may be used to decouple one or more elements from the substrate in a manner such as described in greater detail below.

In a manner such as illustrated in FIG. 1B, second moiety 150 may become coupled to first moiety 130 so as to couple target 160 within each of wells 121, 122, 123, 124. Any suitable moieties that covalently or noncovalently interact with one another may be provided for first and second moieties 130, 150. In one nonlimiting example, first moiety 130 includes streptavidin and second moiety 150 includes biotin. In another nonlimiting example, first moiety 130 includes biotin and second moiety 150 includes streptavidin. Alternatively, target 160 may be coupled within each of the wells 121, 122, 123, 124 through any suitable interactions such as NTA-His-Tag, Spytag-Spycatcher, hybridization of an oligonucleotide to a complementary oligonucleotide, copper(I)-catalyzed click reaction, or strain-promoted azide-alkyne cycloaddition, optionally including a cleavable moiety such as 8-oxo-G or a protein which may be cleaved using a proteinase. Although for simplicity, a single first moiety 130, a single second moiety 150, and a single target 160 are illustrated as being within each of the wells, it will be appreciated that any suitable number of first and second moieties and targets may be coupled within each of the wells, and that each well may have the same number or a different number of moieties or targets coupled therein. In examples such as illustrated in FIG. 1B, capture primers 111, 112 may be coupled to the wells 121, 122, 123, 124 separately from target 160.

In a manner such as illustrated in FIG. 1C, system 100 may include a fluid including a plurality of aptamer candidates 171, 172, 173, 174 and contacting each of the wells 121, 122, 123, 124. Each of the aptamer candidates 171, 172, 173, 174 may include adapter(s) 181, 182 via which that aptamer candidate respectively may be coupled to capture primer 111 or 112 for amplification in a manner such as described below with reference to FIG. 1H. Any aptamer candidate that is selective for the target becomes coupled to target 160. For example, as illustrated in FIG. 1D, aptamer candidate 174 is coupled to target 160 within well 124, while aptamer candidates 171, 172, 173 remain uncoupled to target 160 within wells 121, 122, 123 and may be removed in a manner such as illustrated in FIG. 1E, e.g., by flowing a buffer through wells 121, 122, 123, 124. In some examples, any aptamer candidate that is selective for the target 160 may have a tertiary structure that changes when becoming coupled to the target. As suggested by FIG. 1D, at least some of aptamer candidates 171, 172, 173, 174 may include a hairpin structure including a single stranded loop and single stranded stems that may become coupled to target 160 when the aptamer candidates are selective for that target.

Amplicons may be generated of any aptamer candidate that is selective for the target. For example, generating the amplicons of any aptamer candidate that is selective for the target may include decoupling that aptamer candidate from the target and using PCR to generate the amplicons using that aptamer candidate. For example, aptamer candidate 174 illustrated in FIG. 1E may be decoupled from target 160, and PCR performed to generate amplicons of that aptamer candidate. Such decoupling between aptamer candidate 174 and target 160 may be performed using any suitable reaction condition(s) that disrupt interactions between the target and aptamer, such as heat or buffer exchange in which the salt concentration or pH is made to be unfavorable to the aptamer's tertiary structure or to the forces that stabilize the aptamer-target interface. In some examples, apparatus 100 may include an amplification chamber that is distinct from the wells 121, 122, 123, 124 to perform the PCR. Then, in a manner such as illustrated in FIG. 1F, target 160 in each of wells 121, 122, 123, 124 is contacted with a fluid including amplicons 174 of aptamer candidate 174.

Note that amplicons 174 and aptamer candidate 174 are referred to herein using the same reference numeral because they are equivalent structures; for example, amplicons 174 also are aptamer candidates for target 160 and are expected to become coupled thereto similarly as aptamer candidate 174. In a manner similar to that described with reference to FIG. 1D, any amplicon 174 that is selective for the target becomes coupled to the target, e.g., such as shown in FIG. 1G in which a plurality of amplicons 174 are coupled to respective targets 160. Note, however, that the additional operations for decoupling aptamer candidates from the target and using PCR to generate the amplicons using that aptamer candidate may be omitted.

Apparatus 100 then may be used to sequence aptamer candidate 174 (including any amplicons thereof). For example, any aptamer candidates may be sequenced that remain coupled to target 150 after removing aptamer candidates that are not coupled to the target 160. Aptamer candidates 174 (including any amplicons thereof) illustrated in FIG. 1G may be decoupled from target 160 in a manner such as described with reference to FIG. 1E, and cluster amplification may be used to generate amplicons of aptamer candidates 174, including to generate additional amplicons of any amplicon that is selective for the target. For example, as noted above with reference to FIG. 1A, capture primers 111, 112 may be disposed within wells 121, 122, 123, 124. Capture primer 111 and capture primer 112 may be orthogonal to one another, e.g., capture primer 111 may include a P5 primer and capture primer 112 may include a P7 primer. The capture primers 111, 112 may be coupled to any aptamer candidates (including any amplicons thereof) 174 that remain after any aptamer candidates (and amplicons) are removed that did not become coupled to a target 160. For example, as noted above with reference to FIG. 1A, aptamer candidates 171, 172, 173, 174 may include adapter(s) 181, 182 via which that aptamer candidate respectively may be coupled to capture primer 111 or 112 for amplification.

In a manner such as illustrated in FIG. 1I, adapters 181 of aptamer candidates 174 (or amplicons thereof) may be coupled to capture primers 111, and adapters 182 of the aptamer candidates may be coupled to capture primers 112. Bridge amplification, or other suitable surface-based amplification process, may be used to generate clusters of amplicons 174, 174′ of aptamer candidate (or amplicon) 174 in a manner such as illustrated in FIG. 1I. Note that second moiety 150 and target 160 are omitted from FIG. 1I for simplicity, and either may remain in the well or may be removed, e.g., by cleaving the linker tethering target 160 to moiety 130 or the linker cleaving moiety 130 to the surface. Detection circuitry 190 may be to sequence the amplicons 174, 174′ coupled to the capture primers, e.g., using sequencing-by-synthesis in a manner such as known in the art. Illustratively, a polymerase may be used to extend primer 111 or 112 using labeled (e.g., fluorescently labeled) nucleotides based on the sequence of a respective amplicon 174, 174′, and detection circuitry 190 may identify the sequence in which such nucleotides are added using signals generated by the labels of those nucleotides. Aptamer candidates (or amplicons thereof 174) that are sequenced may be deemed to be aptamers for target 160, for example, because such aptamer candidates had been coupled to target 160 through multiple operations, indicating selectivity for the target.

As noted above with reference to FIG. 1A, the target for the aptamer selection process may be coupled within the wells in any suitable manner, such as using capture primers. For example, FIGS. 2A-2F schematically illustrate an example apparatus and operations used in an alternative process flow for selecting aptamers using sequencing. In the example illustrated in FIG. 2A, the first moiety is coupled within each of the wells by a capture primer, e.g., first moiety 230 is coupled to substrate 210 via capture primer 211, while first moiety 230′ is coupled to substrate 210 via capture primer 212. Capture primer 211 and capture primer 212 may be orthogonal to one another, e.g., capture primer 211 may include a P5 primer and capture primer 212 may include a P7 primer. First moieties 230, 230′ may be contacted with a fluid including molecules 140 including second moiety 150 and target 160 in a manner such as described with reference to FIG. 1A. As illustrated in FIG. 2B, a second moiety 150 may become coupled to first moiety 230 so as to couple target molecule 160 to substrate 210 via capture primer 211 (e.g., within a well, not specifically illustrated), while another second moiety 150 may become coupled to first moiety 230′ so as to couple target molecule 160 to substrate 210 via capture primer 212 (e.g., within the same well or another well, not specifically illustrated). As illustrated in FIG. 2C, aptamer candidates (or amplicons thereof) 174 that are selective for target 160 may be coupled thereto, e.g., in a manner such as described with reference to FIGS. 1D and 1G. As illustrated in FIG. 2D, the aptamer candidates 174 may be decoupled from target 160 for amplification in a manner such as described with reference to FIG. 1E.

In some examples, first moiety 230, 230′ may be detachable from the capture primer 211, 212. As such, in a manner such as illustrated in FIG. 2E, first moieties 230, 230′, second moieties 150, and targets 160 may be decoupled from substrate 210 by detaching first moiety 230, 230′ from its respective capture primer 211, 212. Illustratively, capture primers 211, 212 may include a cleavable moiety such as 8-oxo-G that may be cleaved to detach first moiety 230, 230′, and any elements coupled thereto, prior to performing sequencing. For example, as illustrated in FIG. 2F, adapters 181, 181′ of aptamer candidate 174 and its amplicon 174′ may be hybridized to respective capture primers 211, and adapters 182, 182′ of the aptamer candidate and its amplicon may be hybridized to respective capture primers 212 for amplification and sequencing such as described with reference to FIGS. 1H-1I. Alternatively, in examples such as provided throughout the present disclosure, any suitable ones of target 160, linker(s), first moieties, and second moieties may include proteins that may be digested, and thus decoupled from the substrate and/or capture primers, using a proteinase.

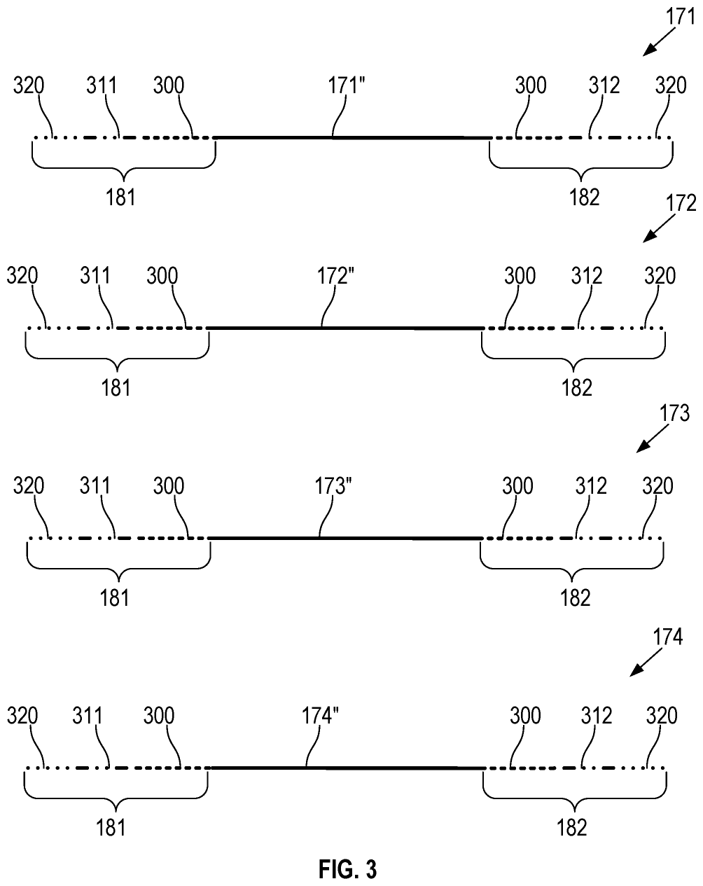

It will be appreciated that the aptamer candidates with which a target is contacted may include any suitable sequence and components. For example, FIG. 3 schematically illustrates example aptamer candidates for use in an apparatus or process flow such as described with reference to FIGS. 1A-1I or 2A-2F. In the nonlimiting example shown in FIG. 3, aptamer candidates 171, 172, 173, 174 each includes an oligonucleotide subsequence (region) 171″, 172″, 173″, 174″ that potentially may be selective for the target, as well as adapters 181 and 182 coupled to the oligonucleotide subsequence. Oligonucleotide subsequences 171″, 172″, 173″, 174″ may be different than one another. Each adapter 181 may include optional spacer 300, capture primer adapter 311 that is complementary to capture primer 111, and optional PCR adapter 320 that may be used to perform PCR amplification of the aptamer candidate if appropriate and if such operations are included in the process flow. Similarly, each adapter 182 may include optional spacer 300, capture primer adapter 312 that is complementary to capture primer 112, and optional PCR adapter 320 that may be used to perform PCR amplification of the aptamer candidate if appropriate and if such operations are included in the process flow. Spacers 300 may provide a suitable distance between the oligonucleotide subsequence 171″, 172″, 173″, 174″ and capture primer adapter 311 or 312 such that capture primer adapter 311 may not inhibit the oligonucleotide subsequence from suitably coupling to target 160. Illustratively, spacer 300 may include 5 or more nucleotides, 10 or more nucleotides, or 15 or more nucleotides.

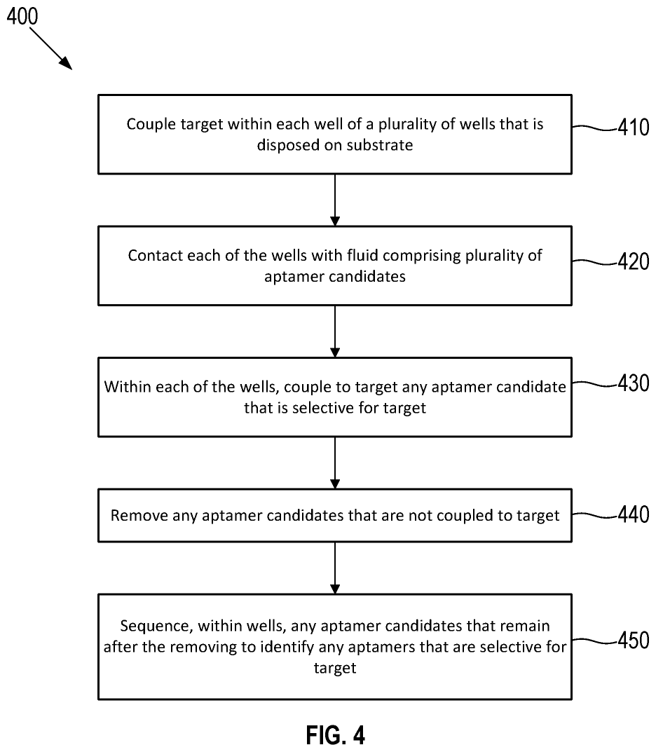

It will be appreciated that any suitable systems, methods, and compositions may be used to select aptamers based on the present teachings. For example, FIG. 4 schematically illustrates example operations in a process flow for selecting aptamers using sequencing. Method 400 illustrated in FIG. 4 includes coupling a target within each well of a plurality of wells that is disposed within a substrate (operation 410). For example, target 160 described with reference to FIG. 1A may be coupled directly to the substrate within each well, e.g., via a suitable linker such as a capture primer in a manner such as described with reference to FIG. 2A. Or, for example, target 160 may be coupled indirectly to the substrate within each well via a first moiety coupled to the substrate and a second moiety that is coupled to the target and that becomes coupled to the first moiety, in a manner such as described with reference to FIGS. 1A-1B. Method 400 illustrated in FIG. 4 also includes contacting each of the wells with a fluid comprising a plurality of aptamer candidates (operation 420). For example, the wells may be contacted with an aptamer library 171, 172, 173, 174 in a manner such as described with reference to FIG. 1C, or may be contacted with amplicons of an aptamer 174 in a manner such as described with reference to FIG. 1F. Method 400 illustrated in FIG. 4 includes, within at least one of the wells, coupling to the target any aptamer candidate that is selective for the target (operation 430). For example, aptamer candidate 174 (or any amplicon thereof) may become coupled to target 160 in a manner such as described with reference to FIG. 1D, 1G, or 2C. Method 400 illustrated in FIG. 4 includes removing any aptamer candidates that are not coupled to the target (operation 440). For example, in a manner such as described with reference to FIG. 1E, aptamer candidates 171, 172, 173 may be removed by flowing buffer through the wells, whereas aptamer candidate 174 remains coupled to target 160 despite such flow. Method 400 illustrated in FIG. 4 includes sequencing, within the wells, any aptamer candidates that remain after the removing to identify any aptamers that are selective for the target (operation 450). For example, amplicons of aptamer candidate 174 (or amplicons thereof) may be generated, and such amplicons sequenced.

### ADDITIONAL COMMENTS

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

While various illustrative examples are described above, it will be apparent to one skilled in the art that various changes and modifications may be made therein without departing from the invention. The appended claims are intended to cover all such changes and modifications that fall within the true spirit and scope of the invention.

What is claimed is:

1. A method of selecting an aptamer from a plurality of aptamer candidates, the method comprising:
coupling a target within each well of a plurality of wells that is disposed within a substrate;
contacting each of the wells with a fluid comprising a plurality of aptamer candidates;
within at least one of the wells, coupling to the target any aptamer candidate that is selective for the target;
removing any aptamer candidates that are not coupled to the target; and
sequencing, within the wells, any aptamer candidates that remain after the removing to identify any aptamers that are selective for the target.

2. The method of claim 1, wherein coupling the target within each well of the plurality of wells comprises:
coupling a first moiety within each of the wells;
coupling a plurality of second moieties to respective ones of the target; and
coupling the second moiety to the first moiety within each of the wells.

3. The method of claim 2, wherein the first moiety comprises streptavidin and the second moiety comprises biotin.

4. The method of claim 2, wherein the first moiety is coupled within each of the wells by a capture primer.

5. The method of claim 4, comprising detaching the first moiety from the capture primer before performing the sequencing.

6. The method of claim 1, wherein the substrate comprises detection circuitry used to sequence the aptamer candidates that remain after the removing.

7. The method of claim 1, wherein the plurality of wells comprise flow cells through which the fluid is flowed in parallel.

8. The method of claim 1, wherein any aptamer candidate that is selective for the target has a tertiary structure that changes when becoming coupled to the target.

9. The method of claim 1, further comprising generating amplicons of any aptamer candidate that is selective for the target.

10. The method of claim 9, wherein generating amplicons of any aptamer candidate that is selective for the target comprises:
decoupling that aptamer candidate from the target; and
using polymerase chain reaction (PCR) to generate the amplicons using that aptamer candidate.

11. The method of claim 10, wherein the PCR is performed in an amplification chamber that is distinct from the wells.

12. The method of claim 9, further comprising:
contacting each of the wells with a fluid comprising the amplicons;
within each of the wells, coupling to the target any amplicon that is selective for the target; and
removing any amplicons are not coupled to the target.

13. The method of claim 12, wherein any aptamer candidates that are sequenced comprise any amplicons that remain after removing any amplicons that are not coupled to the target.

14. The method of claim 12, further comprising generating additional amplicons of any amplicon that is selective for the target.

15. The method of claim 1, wherein sequencing any aptamer candidates comprises:
coupling, to capture primers disposed within the wells, any aptamer candidates that remain after the removing;
performing amplification within the wells to generate amplicons coupled to the capture primers; and
sequencing the amplicons coupled to the capture primers.

16. The method of claim 15, wherein the target is coupled within the wells via respective ones of the capture primers.

17. The method of claim 16, wherein the capture primers couple a first moiety within the wells, and a second moiety is coupled to the first moiety and to the target within the wells.

18. The method of claim 15, wherein the capture primers are coupled to the well separately from the target.

19. The method of claim 15, wherein each of the aptamer candidates comprises first and second adapters that are complementary to respective capture primers.

20. The method of claim 19, wherein each of the aptamer candidates further comprises a first spacer disposed between the first adapter and a region that is a candidate to be selective for the target, and a second spacer disposed between the second adapter and the region that is a candidate to be selective for the target.

21. The method of claim 1, wherein each of the aptamer candidates comprises an oligonucleotide.

22. A system for selecting an aptamer from a plurality of aptamer candidates, the system comprising:
a substrate comprising a plurality of wells;
a target coupled within each of the wells;
a fluid comprising a plurality of aptamer candidates and contacting each of the wells, wherein any aptamer candidate that is selective for the target becomes coupled to the target; and
detection circuitry to sequence any aptamer candidates within the wells to identify any aptamers that are selective for the target.

23.-42. (canceled)
